(19) United States
(12) Patent Application Publication
(10) Pub. No.: US 2009/0186814 A1
(43) Pub. Date: Jul. 23, 2009

(54) Methods and Compositions for Reducing Ischemia-Derived Microvascular Damage

(76) Inventors: Fumiaki Ikeno, Menlo Park, CA (US); Koichi Inagaki, Palo Alto, CA (US); Daria Mochly-Rosen, Menlo Park, CA (US); Mehrdad Rezaee, Los Altos, CA (US)

Correspondence Address: PERKINS COIE LLP, P.O. BOX 1208, SEATTLE, WA, 98111-1208, US

(21) Appl. No.: 11/883,079

(22) PCT Filed: Jan. 26, 2005
(86) PCT No.: PCT/US2005/016114

§ 371 Date: Nov. 18, 2008

Related U.S. Application Data
(60) Provisional application No. 60/647,664, filed on Jan. 26, 2005.

(51) Int. Cl. A61K 38/17; A61K 38/08; A61K 38/10; A61P 9/00; A61P 9/10

(52) U.S. Cl. 514/12; 514/15; 514/14

(57) ABSTRACT

Methods of decreasing the extent of occlusion in the lumen of a mammalian blood vessel due to an ischemic or other hypoxic event are provided. In one form, a method includes administering to a patient in need thereof a pharmaceutically effective amount of an inhibitor of δ protein kinase C, either alone or in combination with a second therapeutic agent, and wherein the blood vessel is a blood vessel of the microvasculature. Additionally, methods of decreasing endothelial cell swelling in a mammalian blood vessel due to an ischemic or other hypoxic event are also provided. In one form, a method includes administering to a patient in need thereof a pharmaceutically effective amount of an inhibitor of δ protein kinase C, either alone or in combination with a second therapeutic agent.

### STATEMENT REGARDING FEDERALLY SPONSORED RESEARCH

This invention was made with government support under grant number 52141 awarded by the National Institutes of Health. The Government has certain rights in the invention.

### FIELD OF THE INVENTION

The present invention relates to methods of inhibiting the no-reflow phenomenon occurring, for example, following recanalization of occluded arteries. The present invention more specifically relates to methods of decreasing the extent of occlusion in the lumen of a mammalian blood vessel due to an ischemic or other hypoxic event. The invention further relates to methods of decreasing endothelial cell swelling in a mammalian blood vessel due to an ischemic or other hypoxic event.

### BACKGROUND OF THE INVENTION

Treatment for acute myocardial infarction (AMI) has been improved by limiting the duration of ischemia using either angioplasty or thrombolytics to disrupt occlusions in coronary arteries and establish reperfusion. However, currently there is no treatment to prevent or decrease reperfusion injury, which occurs after these interventions [Braunwald, E. and Kloner, R. A., J. Clin. Invest. 76:1713-1719 (1985]. Following recanalization of an occluded artery, AMI patients demonstrate impaired microvascular flow (also know as the “no-reflow” phenomenon) due to plugging by blood cells, thromboembolic debris, and edema of endothelial and myocardial cells [Kloner, R. A. et al, J. Clin Invest. 54:1496-1508 (1974); Reffelmann, T. and Kloner, R. A. Heart 87:162-168 (2002)]. The vascular damage induced by reperfusion injury may cause myocardial damage even after the obstruction to flow is removed [Yellon, D. M. and Baxter, G. F., Heart 83:381-387 (2000); Verma, S. et al., Circulation 105:2332-2336 (2002).

There is therefore a need for methods and compositions for decreasing the extent of occlusion in the microvasculature and for decreasing endothelial cell swelling in vessels including the microvasculature. The present invention addresses these needs.

### SUMMARY OF THE INVENTION

It has been discovered that selected isozymes of protein kinase C (PKC) inhibit the no-reflow phenomenon occurring following, for example, recanalization of occluded arteries after an ischemic or other hyopoxic event, thereby decreasing injury to the microvasculature due to such events, including reducing injury due to reperfusion of the affected vessel. It has further been discovered that the above-mentioned regulators of PKC activity decrease the extent of occlusion, such as plugging by blood cells, in the microvasculature of a mammal and endothelial cell swelling as a result of an ischemic or other hypoxic event in the microvasculature. Accordingly, methods of decreasing the extent of occlusion in the lumen of a mammalian blood vessel due to an ischemic or other hypoxic event are provided. Additionally, methods of decreasing endothelial cell swelling in a mammalian blood vessel due to an ischemic or other hypoxic event are also provided.

In one aspect of the invention, methods of decreasing the extent of occlusion in the lumen of a mammalian blood vessel due to an ischemic or other hypoxic event are provided. In one form, a method includes administering to a patient in need thereof a therapeutically effective amount of an inhibitor of δ protein kinase C, wherein the blood vessel is a blood vessel of the microvasculature.

In certain forms of the invention, the patient may be further treated with a therapeutically effective amount of a second therapeutic agent, either together with the inhibitor in a composition or separately.

In a second aspect of the invention, methods of decreasing endothelial cell swelling in a mammalian blood vessel due to an ischemic or other hypoxic event are also provided. In one form, a method includes administering to a patient in need thereof a therapeutically effective amount of an inhibitor of δ protein kinase C. Both the microvasculature and theacrovasculature may be advantageously treated according to the methods of the invention. In yet other forms of the invention, the patient may be further treated with a therapeutically effective amount of a second therapeutic agent, either together with the inhibitor in a composition or separately.

In a third aspect of the invention, methods of inhibiting the no-reflow phenomenon following an ischemic event are provided. In one form, a method includes administering to a patient in need thereof a therapeutically effective amount of an inhibitor of δ protein kinase C. The damage from such an event is independent of the cell-damaging events that occurred in the macrovasculature.

It is an object of the invention to provide methods for inhibiting the no-reflow phenomenon that occurs, for example, following recanalization of occluded arteries.

It is another object of the invention to provide methods of decreasing the extent of occlusion in the lumen of a mammalian blood vessel due to an ischemic or other hypoxic event.

It is a further object of the invention to provide methods of decreasing endothelial cell swelling in a mammalian blood vessel due to an ischemic or other hypoxic event.

These and other objects and advantages of the present invention will be apparent from the descriptions herein.

### BRIEF DESCRIPTION OF THE FIGURES

FIG. 1A shows a view of a transverse section of isolated perfused mouse heart from mice subjected to ischemia and treated with δV1-1 as more fully described in Example 1. WT, wild type mouse; TG, transgenic mouse.

FIG. 1B shows a graph of infarct size as a function of treatment in wild type (WT) or transgenic mice (TG) subjected to acute myocardial infarction and treated with δV1-1 as more fully described in Example 1. *P<0.0I vs. WT; n =5.

FIG. 1C is a graph of total creatine phosphokinase (CPK) release as a function of treatment in transgenic (TG) and wild type (WT) mice subjected to acute myocardial infarction and treated with δV1-1 as more fully described in Example 1. *P<0.01 vs. WT; n=5.

FIG. 1D is a graph of coronary vascular resistance (CVR) as a function of time after reperfusion in wild type (WT) mice or transgenic mice (TG) subjected to acute myocardial infarction and treated with δV1-1 as more fully described in Example 1. †P<0.05 vs. TG; n=5.

FIG. 1E is a graph of TUNEL positive endothelial cells (EC) and myocytes (MC) in wild type ice and transgenic mice subjected to acute myocardial infarction and treated with δV1-1 as more fully described in Example 1. Density of TUNEL-positive nuclei from each group is presented as a percentage of the total numbers of nuclei. Original magnifications are ×200. * P<0.05 vs. EC with vehicle in WT, †P<0.05 vs. MC with vehicle in WT; †P<0.05 vs. MC with vehicle in WT n=5 for each group.

FIG. 1F shows representative fields with Terminal deoxynucleotidyl transferase-mediated dUTP nick-end labeling (TUNEL) staining (yellow) in cardiac tissue in mouse hearts subjected to acute myocardial infarction and treated with δV1-1 as more fully described in Example 1. Myocytes were identified by anti-α-actinin antibody (blue; top), endothelial cells by anti-PECAM-1 (blue; bottom), and nuclei were counterstained with DAPI (green). V, blood vessel.

FIG. 2A shows representative recordings of the Doppler signal at baseline and following intracoronary adenosine administration that causes vasodilation (hyperemia) in pigs subjected to ischemia and treated with δV1-1 as more fully described in Example 2. S and D indicate the state of systolic phase and diastolic phase, respectively. CRF, coronary flow reserve.

FIG. 2B shows a graph of coronary flow reserve (CFR) in the left anterior descending artery (LAD) as a function of time after treatment of pigs with adenosine as more fully described in Example 2. control (open circle); δV1-1-treated hearts (closed circle) (*P<0.01 vs. control; n=9 for each group).

FIG. 2C shows a graph of coronary flow reserve (CFR) in the left anterior descending artery (LAD) as a function of time after treating pigs with by bradykinin as more fully described in Example 2. CFR was assessed before ischemia and at 24 hours (*P<0.01 vs. control; n=6 for each group).

FIG. 2D shows a graph of the ejection fraction (percentage) as a function of time in pigs subjected to ischemia and treated as more fully described in Example 2. For ejection fraction (EF) at each time point, δV1-1-treated hearts were compared to control (*P<0.05 vs. control; n=9 for each group).

FIG. 2E is a graph of hypokinetic area as a function of time in pigs subjected to ischemia and treated as more fully described in Example 2. At each time point, δV1-1-treated hearts were compared to control (*P<0.05 vs. control; n=9 for each group).

FIG. 2F shows a graph of the relationship between infracted area and coronary flow reserve (CFR) in the left anterior descending artery (LAD) of pigs subjected to ischemia and treated with dVl-1 as more fully described in Example 2. There were significant correlations between CFR at 5 days and infarct size inversely (r=−0.49, P<0.05, n=18) and EF at 10 days (r=0.7, P<0.01, n=18).

FIG. 2G shows a graph of the relationship between ejection fraction and the coronary flow reserve (CFR) in the left anterior descending artery (LAD) of pigs subjected to ischemia and treated with dV1-1 as more fully described in Example 2. There were significant correlations between CFR at 5 days and infarct size inversely (r=−0.49, P<0.05, n=18) and EF at 10 days (r=0.7, P<0.01, n=18).

FIG. 3A shows a schematic of δV1-1 treatment (center panel) decreased infarct size as compared to control hearts (left panel; white) in a porcine model of acute myocardial infarction as more fully described in Example 3. Tissue samples (green) were taken from area at risk (red) and non-ischemic area (right panel; blue).

FIG. 3B shows representative fields with TUNEL staining (yellow) shown in heart sections from pigs subject to an acute myocardial infraction and treated with vehicle (control) or δV1-1 as more fully described in Example 3. Myocytes (MC) were identified by anti-α-actinin (blue; top), vascular endothelia3l cells (EC) by anti-PECAM-1 (blue; bottom), and nuclei were counterstained with DAPI (green). (X100-bottom and x400-top; *P<0.05 vs. EC of control, †P<0.05 vs. MC of control, and ‡P<0.05 vs. EC of δV1-1; n=3 for each group); V, blood vessel.

FIG. 3C is a bar graph showing the percentage of TUNEL positive endothelia cells and myocytes in control pigs or pigs treated with δV1-1 as more fully described in Example 3.

FIGS. 3D-3G show representative electron micrographs showing the ultrastructure of endothelial cells and myocytes from control pig hearts subjected to ischemia/reperfusion as more fully described in Example 3. D) Capillary shows red blood cell (arrowhead) and white blood cell (arrow) plugging and nuclear chromatin condensation and margination; E) Another capillary shows endothelial swelling and nuclear chromatin condensation and margination; F) Myocyte has nuclear with variable density chromatin condensation and margination. Mitochondria swelling, fragmentation of the cristae, and intramitochondrial amorphous dense bodies are present (arrowhead); Myocyte also has contraction bands (arrow) and myofilaments are partially distorted. Magnification of electron micrographs was ×1000 to ×6000;

FIGS. 3H-3I show representative electron micrographs showing the ultrastructure of endothelial cells and myocytes from a pig heart subjected to ischemia/reperfusion and treated with δV1-as more fully described in Example 3. Capillary has slight endothelial swelling and slight condensation of chromatin and margination, but the microvasculature lumen is patent; Myocytes have neither contraction bands nor swollen mitochondria (black arrowhead).

### DESCRIPTION OF THE PREFERRED EMBODIMENT

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention provides methods of decreasing injury to the microvasculature derived from an ischemic or other hypoxic event in a mammal. It has been discovered that selected isozymes of protein kinase C (PKC) decrease injury to the microvasculature due to an ischemic or other hypoxic event, including reducing injury due to reperfusion of the affected vessel. It has further been discovered that the above-mentioned regulators of PKC activity decrease the extent of occlusion in the microvasculature of a mammal and endothelial cell swelling as a result of an ischemic or hypoxic event in the microvasculature. “Ischemia”, or “ischemic event”, as used herein refers to an insufficient supply of blood to a specific cell, tissue or organ. A consequence of decreased blood supply is an inadequate supply of oxygen and nutrients to the cell, tissue or organ. By “hypoxic event” or “hypoxia”, it is meant herein an event which causes a cell, tissue or organ to receive an inadequate supply of oxygen. By “microvasculature” it is meant herein blood vessels having an internal diameter of no more than about 50 μm, including the capillaries, arterioles and venules. By “reperfusion” it is meant herein a return of fluid flow to a cell, tissue or organ after a period of no flow or reduced flow. For example, in reperfusion of the heart, fluid or blood returns to the heart through the coronary arteries after occlusion of these arteries have been alleviated.

It has always been assumed by clinicians that damage to the microvasculature resulted predominantly from thrombi breaking off and becoming lodged in the microvasculature, thereby impeding blood flow upon reperfusion. However, it has been discovered herein that decreased blood flow and subsequent and/or continued damage to the microvasculature can occur in the absence of thrombi becoming lodged in the microvasculature. For example, when occlusion and subsequent ischemia is induced with an angioplasty balloon as described herein, decreased blood flow and subsequent damage to the microvasculature occurs as further described in the Examples described herein. Although not being limited by theory, it is believed herein that occlusion of the microvasculature due to an ischemic event, or due to reperfusion of affected macrovasculature after an ischemic event, leading to decreased blood flow and subsequent cell, tissue or organ damage is brought about by a variety of factors. Such factors include plugging or occlusion of the microvasculature with, for example, blood cells, including leukocytes and erythrocytes and apoptotic endothelial cells; and edema of the endothelial cells lining, or otherwise forming the lumen of, capillaries, arterioles and/or venules.

The extent of the damage incurred by the microvasculature from, for example, mechanical shearing of endothelial cells, endothelial cell swelling and occlusion by blood cells is unique to the microvasculature due to the difference in structure and/or size between the microvasculature and macrovasculature. The macrovasculature is composed of a single layer of endothelial cells, whereas the lumen of capillaries of the microvasculature are formed from single endothelial cells folded upon themselves and linked by tight junctions. Arterioles and venules of the microvasculature, although composed of a single layer of endothelia cells as the macrovasculature, are also much smaller than the macrovasculature. Endothelial cell swelling and cellular occlusion can also contribute to damage in the macrovasculature in combination with other factors. However, the damage created by such swelling and occlusion, including the occlusion contributed by the death of endothelial cells and plugging of the microvasculature by blood cells, in the microvasculature can arise in minutes during reperfusion due to the already small lumen formed by the endothelial cells of the microvasculature. It has been discovered herein that, if such swelling and/or occlusion were reduced, especially during reperfusion, the no-reflow phenomenon, and the associated damage to the microvasculature, can be minimized.

Accordingly, in one aspect of the invention, methods for decreasing the extent of occlusion in the lumen of a mammalian blood vessel of the microvasculature derived from an ischemic event, and/or from reperfusion of the macrovasculature and/or microvasculature after an ischemic event, are provided. In one form, a method includes administering to a patient in need thereof a therapeutically effective amount of an inhibitor of δ protein kinase C, wherein said blood vessel is a blood vessel of the microvasculature. As discussed above, such a method will advantageously reduce reperfusion injury. Reperfusion injury, as used herein and as known in the art, refers to injury resulting from restoring blood flow to a blood vessel that experienced, or was otherwise affected by, an ischemic or other hypoxic event. Examples of reperfusion injury include cell, tissue or organ damage or death that result from restoring blood flow to a blood vessel that experienced, or was otherwise affected by, an ischemic or other hypoxic event.

The blood vessels of the microvasculature that may be treated according to the methods of the present invention include the capillaries, arterioles and venules associated with the various systems of the body that may be affected by an ischemic event. The diameter of the lumen of blood vessels of the microvasculature are known to those skilled in the art. For example, the capillaries typically have an inner diameter of about 5 μm to about 10 μm, whereas the arterioles and venules typically have an inner diameter of about 10 μm to about 50 μ. Such blood vessels may have larger inner diameters depending on the circumstance. For example, the blood vessels of the microvasculature may have a lumen with an inner diameter of no greater than about 250 μm. Systems of the body, and the organs associated with such systems, that have microvasculature that may be affected by an ischemic event include, for example, the nervous system, including the brain, spinal chord and peripheral nerves; the respiratory system, including the lungs; the gastrointestinal tract, including the small and large intestines, the musculoskeletal system, including the upper and lower extremities; the genitourinary system; including the kidneys; and the cardiovascular system, including the heart.

One skilled in the art is familiar with the microvasculature of the aforementioned body systems that may be affected by an ischemic event and, in light of the description herein, the microvasculature that may be treated according to the methods of the present invention. For example, with respect to the cardiovascular system, the microvasculature of the heart amenable for treatment includes those vessels that are derived from, or feed into, the coronary arteries, the pulmonary arteries, the aorta, the superior and inferior pulmonary veins, the great cardiac vein, the small cardiac vein, the inferior vena cava, and the superior vena cava. It is understood that this list relating to the heart microvasculature is not an exhaustive list of the blood vessels in which the extent of occlusion may be reduced in the heart and thus is merely illustrative. In light of the disclosure herein, one skilled in the art is aware of all other vessels of the microvasculature, including those connected directly or indirectly to the heart, that may be amenable for treatment to decrease the extent of occlusion in such vessels as described herein.

A wide variety of inhibitors of δPKC may be utilized in the present invention. By inhibitor of δPKC, it is meant herein a compound that inhibits the biological activity or function of δPKC. As known in the art, δPKC is involved a myriad of cellular processes, including regulation of cell growth, and regulation of gene expression. The inhibitors may, for example, inhibit the enzymatic activity of δPKC. The inhibitors may inhibit the activity of δPKC by, for example, preventing activation of δPKC or may prevent binding of δPKC to its protein substrate. Such an inhibition of enzymatic activity would prevent, for example, phosphorylation of amino acids in proteins. The inhibitor may also prevent binding of δPKC to its receptor for activated kinase (RACK), or any other anchoring protein, and subsequent translocation of 8PKC to its subcellular location. In one form of the invention, organic molecule inhibitors, including alkaloids, may be utilized. For example, benzophenanthridine alkaloids may be used, including chelerythrine, sanguirubine, chelirubine, sanguilutine, and chililutine. Such alkaloids can be purchased commercially and/or isolated from plants as known in the art and as described, for example, in U.S. Pat. No. 5,133,981.

The bisindolylmaleimide class of compounds may also be used as inhibitors of δPKC. Exemplary bisindolylmaleimides include bisindolylmaleimide I, bisindolylmaleimide II, bisindolylmaleimide III, bisindolylmaleimide IV, bisindolylmaleimide V, bisindolylmaleimide VI, bisindolylmaleimide VII, bisindolylmaleimide VIII, bisindolylmaleimide IX, bisindolylmaleimide X and other bisindolylmaleimides that are effective in inhibiting δPKC. Such compounds may be purchased commercially and/or synthesized by methods known to the skilled artisan and as described, for example, in U.S. Pat. No. 5,559,228 and Brenner, et al., Tetrahedron 44(10) 2887-2892 (1988). Anti-helminthic dyes obtained from the kamala tree and effective in inhibiting δPKC may also be utilized, including rottlerin, and may be purchased commercially or synthesized by the skilled artisan.

In certain forms of the invention, a protein inhibitor of δPKC may be utilized. The protein inhibitor may be in the form of a peptide. Protein, peptide and polypeptide as used herein and as known in the art refer to a compound made up of a chain of amino acid monomers linked by peptide bonds. Unless otherwise stated, the individual sequence of the peptide is given in the order from the amino terminus to the carboxyl terminus. The protein inhibitor of δPKC may be obtained by methods known to the skilled artisan. For example, the protein inhibitor may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla., (1997).

Alternatively, the protein inhibitor may be produced by recombinant technology methods as known in the art and as described, for example, in Sambrook et al., Molecular Cloning: A Laboratory Manual, Cold Springs Harbor laboratory, 2^{nd} ed., Cold Springs Harbor, New York (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. For example, an expression vector may be used to produce the desired peptide inhibitor in an appropriate host cell and the product may then be isolated by known methods. The expression vector may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

The inhibitor may be derived from an isozyme of PKC, such as δV1-1, whose amino acid sequence from Rattus norvegicus is set forth in SEQ ID NO:1 (SFNSYELGSL), representing amino acids 8-17 of rat δPKC as found in Genbank Accession No. AAH76505. Alternatively, the peptide inhibitor may be other fragments of PKC, such as δv1-2, δV1-5 and/or δV5, or some combination of δV1-1, δV1-2, δV1-5 and δV5. The amino acid sequence of δV1-2 from Rattus norvegicus is set forth in SEQ ID NO:2 (ALTTDRGKTLV), representing amino acids 35 to 45 of rat δPKC found in Genbank Accession No. AAH76505. The amino acid sequence of δV1-5 from Rattus norvegicus is set forth in SEQ ID NO:3 (KAEFWLDLQPQAKV), representing amino acids 569 to 626 of rat δPKC found in Genbank Accession No. MH76505. The amino acid sequence of δV5 is set forth in SEQ ID NO:4 (PFRPKVKSPRPYSNFDQEFLNEKARLSYSDKNLIDSMDQSAFAGFSFVNPKFEHLLED), representing amino acids 561-626 of human δPKC found in Genbank Accession No. BAA01381, with the exception that amino acid 11 (aspartic acid) is substituted with a proline.

The peptide inhibitors may include natural amino acids, such as the L-amino acids or non-natural amino acids, such as D-amino acids. The amino acids in the peptide may be linked by peptide bonds or, in modified peptides described herein, by non-peptide bonds.

A wide variety of modifications to the amide bonds which link amino acids may be made and are known in the art. Such modifications are discussed in general reviews, including in Freidinger, R. M. “Design and Synthesis of Novel Bioactive Peptides and Peptidomimetics” J. Med. Chem. 46:5553 (2003), and Ripka, A. S., Rich, D. H. “Peptidomimetic Design” Curr. Opin. Chem. Biol. 2:441 (1998). These modifications are designed to improve the properties of the peptide by increasing the potency of the peptide or by increasing the half-life of the peptide.

The potency of the peptide may be increased by restricting the conformational flexibility of the peptide. This may be achieved by, for example, including the placement of additional alkyl groups on the nitrogen or alpha-carbon of the amide bond, such as the peptoid strategy of Zuckerman et al, and the alpha modifications of, for example Goodman, M. et. al. [Pure Appl. Chem. 68:1303 (1996)]. The amide nitrogen and alpha carbon may be linked together to provide additional constraint [Scott et al, Org. Letts. 6:1629-1632 (2004)].

The half-life of the peptide may be increased by introducing non-degradable moieties to the peptide chain. This may be achieved by, for example, replacement of the amide bond by a urea residue [Patil et al, J. Org. Chem. 68:7274-7280 (2003)] or an aza-peptide link [Zega and Urleb, Acta Chim. Slov. 49:649-662 (2002)]. Other examples of non-degradable moieties that may be introduced to the peptide chain include introduction of an additional carbon [“beta peptides”, Gellman, S. H. Acc. Chem. Res. 31:173 (1998)] or ethene unit [Hagihara et al, J. Am. Chem. Soc. 114:6568 (1992)] to the chain, or the use of hydroxyethylene moieties [Patani, G. A., Lavoie, E. J. Chem. Rev. 96:3147-3176 (1996)] and are also well known in the art. Additionally, one or more amino acids may be replaced by an isosteric moiety such as, for example, the pyrrolinones of Hirschmann et al [J. Am. Chem. Soc. 122:11037 (2000)], or tetrahydropyrans [Kulesza, A. et al., Org. Letts. 5:1163 (2003)].

Although the peptides are described primarily with reference to amino acid sequences from Rattus norvegicus, it is understood that the peptides are not limited to the specific amino acid sequences setforth in SEQ ID NOS:1-4. Skilled artisans will recognize that, through the process of mutation and/or evolution, polypeptides of different lengths and having different constituents, e.g., with amino acid insertions, substitutions, deletions, and the like, may arise that are related to, or sufficiently similar to, a sequence set forth herein by virtue of amino acid sequence homology and advantageous functionality as 35 described herein. The terms “δV1-1 peptide”, “δV1-2 peptide”, “δV1-5 peptide” and “δV5 peptide” are used to refer generally to the peptides having the features described herein and preferred examples include peptides having the amino acid sequence of SEQ ID NO:1, SEQ ID NO:2, SEQ ID NO:3 and SEQ ID NO:4, respectively. Also included within this definition, and in the scope of the invention, are variants of the peptides which function in decreasing the extent of occlusion in the lumen of a mammalian blood vessel and/or decreasing endothelial cell swelling in a mammalian blood vessel, both as described herein.

The peptide inhibitors described herein also encompass amino acid sequences similar to the amino acid sequences set forth herein that have at least about 50% identity thereto and function to decrease the extent of occlusion in the lumen of a mammalian blood vessel and/or decrease endothelial cell swelling in a mammalian blood vessel, both as described herein. Preferably, the amino acid sequences of the peptide inhibitors encompassed in the invention have at least about 60% identity, further at least about 70% identity, preferably at least about 80% identity, more preferably at least about 90% identity, and further preferably at least about 95% identity, to the amino acid sequences, including SEQ ID NOS:1-4, set forth herein.

Percent identity may be determined, for example, by comparing sequence information using the advanced BLAST computer program, including version 2.2.9, available from the National Institutes of Health. The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen, Computers and Chemistry 17:149-163 (1993).

Accordingly, fragments or derivatives of peptide inhibitors described herein may also be advantageously utilized that include amino acid sequences having the specified percent identities to SEQ ID NOS:1-4 described herein to reduce the extent of occlusion in the lumen of a mammalian blood vessel and/or to reduce endothelial cell swelling in a mammalian blood vessel, both as described herein. For example, fragments or derivatives of δV1-1, δV1-2, δV1-5 and δV5 that are effective in inhibiting δPKC and decreasing the extent of occlusion in the lumen of a mammalian blood vessel and/or decreasing endothelial cell swelling in mammalian blood vessel, both as described herein, may also advantageously be utilized in the present invention.

Conservative amino acid substitutions may be made in the amino acid sequences to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, aspartic acid, glutamic acid and their amides, are also considered interchangeable herein.

Accordingly, modifications to δV1-1 that are expected to result in effective inhibition of δPKC and a concomitant reduction in the extent of occlusion in the lumen of a mammalian blood vessel and/or reduction in endothelial cell swelling in a mammalian blood vessel, both as described herein, include the following changes to SEQ ID NO:1 shown in lower case: tFNSYELGSL (SEQ ID NO:5), aFNSYELGSL (SEQ ID NO:6), SFNSYELGtL (SEQ ID NO:7), including any combination of these three substitutions, such as tFNSYELGtL (SEQ ID NO:8). Other potential modifications include SyNSYELGSL (SEQ ID NO:9), SFNSfELGSL (SEQ ID NO:10), SNSYdLGSL (SEQ ID NO:11), SFNSYELpSL (SEQ ID NO:12).

Other possible modifications that are expected to produce a peptide that functions in the invention include changes of one or two L to I or V, such as SFNSYEiGSv (SEQ ID NO:13), SFNSYEvGSi (SEQ ID NO:14), SFNSYELGSv (SEQ ID NO:15), SFNSYELGSi (SEQ ID NO:16), SFNSYEiGSL (SEQ ID NO:17), SFNSYEvGSL (SEQ ID NO:18), aFNSYELGSL (SEQ ID NO:19), any combination of the above-described modifications, and other conservative amino acid substitutions described herein.

Fragments and modification of fragments of δV1-1 are also contemplated, including:

| | |
|---|---|
| YELGSL, | (SEQ ID NO:20) |
| YdLGSL, | (SEQ ID NO:21) |
| fdLGSL, | (SEQ ID NO:22) |
| YdIGSL, | (SEQ ID NO:23) |
| iGSL, | (SEQ ID NO:24) |
| YdvGSL, | (SEQ ID NO:25) |
| YdLpsL, | (SEQ ID NO:26) |
| YdLgiL, | (SEQ ID NO:27) |
| YdLGSi, | (SEQ ID NO:28) |
| YdLGSv, | (SEQ ID NO:29) |
| LGsL, | (SEQ ID NO:30) |
| iGSL, | (SEQ ID NO:31) |
| vGSL, | (SEQ ID NO:32) |
| LpSL, | (SEQ ID NO:33) |
| LGiL, | (SEQ ID NO:34) |
| LGSi, | (SEQ ID NO:35) |
| LGSv. | (SEQ ID NO:36) |

Accordingly, the term “a δV1-1 peptide” as used herein further refers to a peptide identified by SEQ ID NO:1 and to a peptide having an amino acid sequence having the specified percent identity described herein to the amino acid sequence of SEQ ID NO:1, including but not limited to the peptides set forth in SEQ ID NOS:5-19, as well as fragments of any of these peptides that retain activity for reducing the extent of occlusion in the lumen of a mammalian blood vessel and/or reducing endothelial cell swelling, both as described herein, as exemplified by but not limited to SEQ ID NOS:20-36.

Modifications to δV1-2 that are expected to result in effective inhibition of δPKC and a concomitant decrease in the extent of occlusion in the lumen of a mammalian blood vessel and/or decrease in endothelial cell swelling in a mammalian blood vessel, both as described herein include the following changes to SEQ ID NO:2 shown in lower case: ALsTDRGKTLV (SEQ ID NO:37), ALTsDRGKTLV (SEQ ID NO:38), ALTTDRGKsLV (SEQ ID NO:39), and any combination of these three substitutions, ALTTDRpKTLV (SEQ ID NO:40), ALTTDRGrTLV (SEQ ID NO:41), ALTTDkGKTLV (SEQ ID NO:42), ALTTDkGkTLV (SEQ ID NO:43), changes of one or two L to 1, or V and changes of V to 1, or L and any combination of the above. In particular, L and V can be substituted with V, L, I R and D, E can be substituted with N or Q. One skilled in the art would be aware of other conservative substitutions that may be made to achieve other derivatives of δV1-2 in light of the description herein.

Accordingly, the term “a δV1-2 peptide” as further used herein refers to a peptide identified by SEQ ID NO:2 and to a peptide having an amino acid sequence having the specified percent identity described herein to the amino acid sequence of SEQ ID NO:2, including but not limited to the peptides set forth in SEQ ID NOS:37-43, as well as fragments of any of these peptides that retain activity for decreasing the extent of occlusion in the lumen of a mammalian blood vessel and/or decreasing endothelial cell swelling in a mammalian blood vessel, both as described herein.

Modifications to δV1-5 that are expected to result in effective inhibition of 6PKC and a concomitant decrease in the extent of occlusion in the lumen of a mammalian blood vessel and/or decrease in endothelial cell swelling, both as described herein. include the following changes to SEQ ID NO:3 shown in lower case: rAEFWLDLQPQAKV (SEQ ID

| | |
|---|---|
| rAEFWLDLQPQAKV; | (SEQ ID NO:44) |
| KAdFWLDLQPQAKV; | (SEQ ID NO:45) |
| KAEFWLeLQPQAKV, | (SEQ ID NO:46) |
| KAEFWLDLQPQArV, | (SEQ ID NO;47) |
| KAEyWLDLQPQAKV, | (SEQ ID NO:48) |
| KAEFWiDLQPQAKV, | (SEQ ID NO:49) |
| KAEFWvDLQPQAKV, | (SEQ ID NO:50) |
| KAEFWLDiQPQAKV, | (SEQ ID NO:51) |
| KAEFWLDvQPQAKV, | (SEQ ID NO:52) |
| KAEFWLDLnPQAKV, | (SEQ ID NO:53) |
| KAEFWLDLQPnAKV, | (SEQ ID NO:54) |
| KAEFWLDLQPQAKi, | (SEQ ID NO;55) |
| KAEFWLDLQPQAKI, | (SEQ ID NO:56) |
| KAEFWaDLQPQAKV, | (SEQ ID NO:57) |
| KAEFWLDaQPQAKV, | (SEQ ID NO;58) |
| and | |
| KAEFWLDLQPQAKa. | (SEQ ID NO:59) |

Fragments of δV1-5 are also contemplated, including: KAEFWLD (SEQ ID NO:60),

| | |
|---|---|
| KAEFWLD, | (SEQ ID NO:60) |
| DLQPQAKV, | (SEQ ID NO:61) |
| EFWLDLQP, | (SEQ ID NO:62) |
| LDLQPQA, | (SEQ ID NO:63) |
| LQPQAKV, | (SEQ ID NO:64) |
| AEFWLDL, | (SEQ ID NO:65) |
| and | |
| WLDLQPQ. | (SEQ ID NO:66) |

Modifications to fragments of δV1-5 are also contemplated and include the modifications shown for the full-length fragments as well as other conservative amino acid substitutions described herein. The term “a δV1-5 peptide” as further used herein refers to SEQ ID NO:3 and to a peptide having an amino acid sequence having the specified percent identity described herein to an amino acid sequence of SEQ ID NO:3, as well as fragments thereof that retain activity for decreasing the extent of occlusion in the lumen of a mammalian blood vessel and/or decreasing endothelial cell swelling in a mammalian blood vessel, both as described herein.

Modifications to δV5 that are expected to result in effective inhibition of δPKC and a concomitant decrease in the extent of occlusion in the lumen of a mammalian blood vessel and/or decrease in endothelial cell swelling, both as described herein, include making one or more conservative amino acid substitutions, including substituting: R at position 3 with Q; S at position 8 with T; F at position 15 with W; V at position 6 with L and D at position 30 with E; K at position 31 with R; and E at position 53 with D, and various combinations of these modifications and other modifications that can be made by the skilled artisan in light of the description herein.

Fragments of δV5 are also contemplated, and include, for example, the following:

| | |
|---|---|
| SPRPYSNF, | (SEQ ID NO:67) |
| RPYSNFDQ, | (SEQ ID NO:68) |
| SNFDQEFL, | (SEQ ID NO:69) |
| DQEFLNEK, | (SEQ ID NO:70) |
| FLNEKARL, | (SEQ ID NO:71) |
| LIDSMDQS, | (SEQ ID NO:72) |
| SMDQSAFA, | (SEQ ID NO:73) |
| DQSAFAGF, | (SEQ ID NO:74) |
| FVNPKFEH, | (SEQ ID NO:75) |
| KFEHLLED, | (SEQ ID NO:76) |
| NEKARLSY, | (SEQ ID NO:77) |
| RLSYSDKN, | (SEQ ID NO:78) |
| SYSDKNLI, | (SEQ ID NO:79) |
| DKNLIDSM, | (SEQ ID NO:80) |
| PFRPKVKS, | (SEQ ID NO:81) |
| RPKVKSPR, | (SEQ ID NO:82) |
| and | |
| VKSPRPYS. | (SEQ ID NO:83) |

Modifications to fragments of δV5 are also contemplated and include the modifications shown for the full-length fragments as well as other conservative amino acid substitutions described herein. The term “a δV5 peptide” as further used herein refers to SEQ ID NO:4 and to a peptide having an amino acid sequence having the specified percent identity described herein to an amino acid sequence of SEQ ID NO:4, as well as fragments thereof that retain activity for decreasing the extent of occlusion in the lumen of a mammalian blood vessel and/or decreasing endothelial cell swelling in a mammalian blood vessel, both as described herein. The inhibitors used for treatment herein may include a combination of the peptides described herein.

Other suitable molecules or compounds, including small molecules, that may act as inhibitors of 6PKC may be determined by methods known to the art. For example, such molecules may be identified by their ability to translocate δPKC to its subcellular location. Such assays may utilize, for example, fluorescently-labeled enzyme and fluorescent microscopy to determine whether a particular compound or agent may aid in the cellular translocation of δPKC. Such assays are described, for example, in Schechtman, D. et al., J. Biol. Chem. 279(16):15831-15840 (2004) and include use of selected antibodies. Other assays to measure cellular translocation include Western blot analysis as described in Dorn, G. W., II et al., Proc. Natl. Acad. Sci. U.S.A. 96(22):12798-12803 (1999) and Johnson, J. A. and Mochly-Rosen, D., Circ Res. 76(4):654-63 (1995).

The inhibitors may be modified by being part of a fusion protein. The fusion protein may include a protein or peptide that functions to increase the cellular uptake of the peptide inhibitors, has another desired biological effect, such as a therapeutic effect, or may have both of these functions. For example, it may be desirable to conjugate, or otherwise attach, the δV1-1 peptide, or other peptides described herein, to a cytokine or other protein that elicits a desired biological response. The fusion protein may be produced by methods known to the skilled artisan. The inhibitor peptide may be bound, or otherwise conjugated, to another peptide in a variety of ways known to the art. For example, the inhibitor peptide may be bound to a carrier peptide, such as a cell permeable carrier peptide or other peptide described herein via cross-linking wherein both peptides of the fusion protein retain their activity. As a further example, the peptides may be linked or otherwise conjugated to each other by an amide bond from the C-terminal of one peptide to the N-terminal of the other peptide. The linkage between the inhibitor peptide and the other member of the fusion protein may be non-cleavable, with a peptide bond, or cleavable with, for example, an ester or other cleavable bond known to the art.

Furthermore, in other forms of the invention, the cell permeable carrier protein or peptide that may increase cellular uptake of the peptide inhibitor may be, for example, a Drosophila Antennapedia homeodomain-derived sequence which is set forth in SEQ ID NO:84 (CRQIKIWFQNRRMKWKK), and may be attached to the inhibitor by cross-linking via an N-terminal Cys-Cys bond as discussed in Theodore, L., et al. J. Neurosci. 15:7158-7167 (1995); Johnson, J. A., et al. Circ. Res 79:1086 (1996). Alternatively, the inhibitor may be modified by a Transactivating Regulatory Protein (Tat)-derived transport polypeptide (such as from amino acids 47-57 of Tat shown in SEQ ID NO:85; YGRKKRRQRRR) from the Human Immunodeficiency Virus, Type 1, as described in Vives, et al., J. Biol. Chem, 272:16010-16017 (1997), U.S. Pat. No. 5,804,604 and Genbank Accession No. AAT48070; or with polyarginine as described in Mitchell, et al. J. Peptide Res. 56:318-325 (2000) and Rothbard, et al., Nature Med. 6:1253-1257 (2000). The inhibitors may be modified by other methods known to the skilled artisan in order to increase the cellular uptake of the inhibitors.

The inhibitors may be advantageously administered in various forms. For example, the inhibitors may be administered in tablet form for sublingual administration, in a solution or emulsion. The inhibitors may also be mixed with a pharmaceutically-acceptable carrier or vehicle. The vehicle may be a liquid, suitable, for example, for parenteral administration, including water, saline or other aqueous solution, or may be an oil or aerosol. The carrier may be selected for intravenous or intraarterial administration, and may include a sterile aqueous or non-aqueous solution that may include preservatives, bacteriostats, buffers and antioxidants known to the art. In the aerosol form, the inhibitor may be used as a powder, with properties including particle size, morphology and surface energy known to the art for optimal dispersability. In tablet form, a solid carrier may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The inhibitors may also be administered in forms in which other similar drugs known in the art are administered.

The inhibitors may be administered to a patient by a variety of routes. For example, the inhibitors may be administered parenterally, including intraperitoneally, intravenously, intraarterially, subcutaneously, or intramuscularly. The inhibitors may also be administered via a mucosal surface, including rectally, and intravaginally; intranasally, including by inhalation; sublingually; intraocularly and transdermally. Combinations of these routes of administration are also envisioned. A preferred mode of administration is by infusion or reperfusion through the occluded or partially-occluded artery, or an artery that is connected to such an occluded or partially-occluded artery. By “partially-occluded artery” it is meant herein an artery in which blood flow is reduced after an ischemic attack or other hypoxic event affecting the heart blood vessels when compared to blood flow prior to such event or attack. Included in the definition of “partially-occluded artery” is an artery in which blood flow is reduced compared to a baseline or standard blood flow rate for that blood vessel. Such rates are known to the skilled artisan. In certain forms of the invention, the inhibitor described herein may be co-administered in a composition with a second therapeutic agent to decrease endothelial cell swelling in a mammalian blood vessel and/or to decrease the extent of occlusion in the lumen of a mammalian blood vessel due to an ischemic event and/or the reperfusion of a blood vessel affected by an ischemic event. Alternatively, the second therapeutic agent and inhibitor may be administered separately. A wide variety of therapeutic agents are envisioned for treatment, including vasodilators. Exemplary vasodilators that may be included in the compositions of the invention, or which may otherwise be separately administered to a patient, include protein-based vasodilators, including bradykinin; lipid-based vasodilators, including prostacyclin or its synthetic analogs, including iloprost and cisaprost; nicotinic acid, niacin, beta adrenergic blocking drugs, including sotalol, timolol, esmolol, carteolol, carvedilol, nadolol, propranolol, betaxolol, penbutolol), metoprolol, labetalol, acebutolol, (atenolol), metoprolol), labetalol, pindolol, and bisoprolol. Other vasodilators known to the art may also be used.

The amount of inhibitor in the compositions will range from about 1 weight percent to about 99 weight percent, and preferably about 20 weight percent to about 70 weight percent. The amount of vasodilator in the compositions will also range from about 1 weight percent to about 99 weight percent, and preferably about 20 weight percent to about 70 weight percent. Weight percent as defined herein is the amount of the agent in mg divided by 100 grams of the composition.

A therapeutically effective amount of the inhibitor is provided. As used herein, a therapeutically effective amount of the inhibitor is the quantity of the inhibitor required to decrease endothelial cell swelling in a mammalian blood vessel, to decrease the extent of occlusion in the microvasculature of a mammal and/or to otherwise reduce the cell, tissue or organ damage or death that occurs due to reperfusion following recanalization after an ischemic or other hypoxic or cell damaging event. This amount will vary depending on the time of administration (e.g., prior to an ischemic event, at the onset of the event or thereafter), the route of administration, the duration of treatment, the specific inhibitor used and the health of the patient as known in the art. The skilled artisan will be able to determine the optimum dosage. Generally, the amount of inhibitor typically utilized may be, for example, about 0.001 mg/kg body weight to about 3 mg/kg body weight, but is preferably about 0.01 mg/kg to about 0.5 mg/kg.

A therapeutically effective amount of the second therapeutic agent is provided either alone or co-administered as a composition with the inhibitors described herein. This therapeutically effective amount will vary as described above, especially in regard to the nature of the agent. Where the therapeutic agent is a vasodilator, the therapeutically Effective amount of vasodilator is sufficient to dilate blood vessels to increase the internal diameter of the vessels by at least about 10%, preferably by at least about 25%, further preferably by at least about 50%, at least about 75%, more preferably at least about 90% and more preferably at least about 95% or 100% compared to the internal diameter of the blood vessel prior to such treatment, including during the onset of an ischemic event or other event described herein or after a specified time period after the onset of such an event, such as about 24 hours after the onset of the event. This therapeutically effective amount is defined as above for the inhibitor and will vary as described above The skilled artisan can determine the appropriate amount.

The patient to be treated is typically one in need of such treatment, including one that is susceptible to, or has experienced, an ischemic event or other hypoxic event or otherwise has the potential to incur cellular, tissue or organ damage or death as a result of such an event, including during or after reperfusion of the vessel. The patient is furthermore typically a vertebrate, preferably a mammal, and including a human. Other animals which may be treated include farm animals, such as horse, sheep, cattle, and pigs. Other exemplary animals that may be treated include cats, dogs; rodents, including those from the order Rodentia, such as mice, rats, gerbils, hamsters, and guinea pigs; members of the order Lagomorpha, including rabbits and hares, and any other mammal that may benefit from such treatment. The patient is preferably treated in vivo, preferably at the onset of an ischemic or other hypoxic event. The patient may also be treated after about 1 minute to about 10 hours, but preferably between about 1 minute to about 2 hours, and further preferably after no more than about 10 hours, after occurrence of the ischemic or other event leading to hypoxia and/or cellular nutrient deprivation.

In yet another aspect of the invention, methods of decreasing endothelial cell swelling in a mammalian blood vessel caused by an ischemic or other hypoxic event are provided. In one form, a method includes administering to a patient in need thereof a therapeutically effective amount of a protein inhibitor of δ protein kinase C. The methods may advantageously be applied to the both the microvasculature and the macrovasculature. In one form, a method includes administering to a patient in need thereof a therapeutically effective amount of a protein inhibitor of δ protein kinase C.

The blood vessels amenable to treatment wherein endothelial cell swelling may be reduced or which may otherwise benefit from treatment include the microvasculature, including the capillaries, arterioles and venules, of the body systems previously discussed herein. The macrovasculature associated with the body systems previously described herein will also exhibit decreased endothelial cell swelling after treatment according to the methods of the present invention. One skilled in the art is aware of such vessels that will experience decreases in endothelial cell swelling after an ischemic event after being treated according to the methods of the present invention in light of the disclosure herein. Examples of such vessels include, in the heart, the coronary arteries, the pulmonary arteries, the aorta, the superior and inferior pulmonary veins, the great cardiac vein, the small cardiac vein, the inferior vena cava, and the superior vena cava; in the pancreas include the anterior and posterior inferior pancreaticoduodenal arteries, anterior and posterior superior pancreaticoduodenal arteries, and the pancreatic veins; in the duodenum of the small intestine include the superior and inferior pancreaticoduodenal arteries and the portal vein; in the jejunum and ileum of the small intestine include the superior mesenteric artery and superior mesenteric vein; in the large intestine include the ileocolic artery, the appendicular artery; the right, middle and left colic arteries; the superior sigmoid artery, the sigmoid artery, the ileocolic vein, the right colic vein, and the superior and inferior mesenteric veins. It is understood that this list relating to the macrovasculature is not an exhaustive list of the blood vessels in which the extent of endothelial cell swelling may be reduced according to the methods of the present invention and thus is merely illustrative. In light of the disclosure herein, one skilled in the art is aware of all other vessels of the macrovasculature that may be amenable for treatment to decrease endothelial cell swelling therein as described herein. As an example, included in the arteries that may benefit from treatment herein are the arteries from which the aforementioned arteries branch, or are otherwise derived from, and the arteries and branches that the aforementioned arteries drain into or are otherwise connected to. Included in the veins that may benefit from treatment herein are the veins from which the aforementioned veins branch, or are otherwise derived from, and the veins and branches that the aforementioned veins drain into or are otherwise connected to.

Reference will now be made to specific examples illustrating the invention described above. It is to be understood that the examples are provided to illustrate preferred embodiments and that no limitation to the scope of the invention is intended thereby.

### EXAMPLE 1

The Effect of Expression of δV1-1 during reperfusion in hearts of δV1-1 transgenic Mice on Coronary Vascular Resistance, Infarct Size and Apoptosis in Mice Subjected to Global Ischemia

This example shows that transgenic mice expressing δV1-1 exhibited improved coronary vascular resistance, decreased infarct size and decreased apoptosis compared to control mice. Further benefits were observed when the transgenic mice were exogenously treated with δV1-1.

### Methods

All animal studies were approved by Stanford's Institutional Animal Care and Use Committee.

### Ex Vivo Model of Global Ischemia and Reperfusion Injury Using δV1-1 Transgenic Mouse Hearts

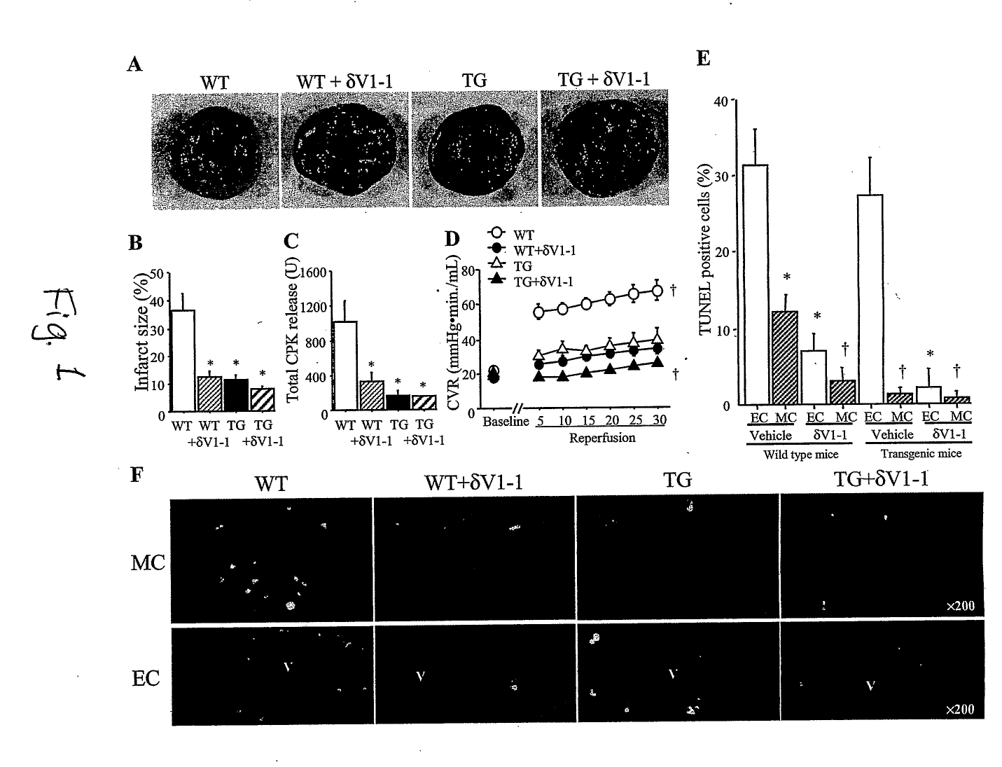

Transgenic mice (TG) that selectively express δV1-1 in myocytes were created using α- myosin heavy chain promoter. Hah, H. S., et al., Circ. Res. 91:741-748 (2002). Hemodynamic and morphometric parameters in these transgenic mice, as measured by echocardiographic measurements in vivo, were not different from those measured in wild type mice (WT). Inagaki, K., et al., Circulation 108:869-875 (2003). Mice were heparinized (4000U/kg IP) and anesthetized with sodium pentobarbital (200mg/kg IP). Hearts were perfused with an oxygenated Krebs-Henseleit buffer at 37° C. in a Langendorff system as previously described in Inagaki, K., et al., Circulation 108:869-875 (2003). Hearts were subjected to a 30-minute global ischemia and a 120-minute full reperfusion. The coronary flow rate was kept constant at 3 mL/minute (0.04L/min/g; initial coronary perfusion pressure: WT 67.4±5.1, WT+δV1-1 61.0±4.6, TG 62.6±3.9, TG+δV1-1 64.6±5.4 mmHg; PENS, n=5 for each group; FIG. 1) using an adjustable-speed rotary pump during the experiment to provide 60-80 mmHg of initial coronary perfusion pressure (CPP) as previously reported in Webster, K. A., et al., J. Clin. Invest. 104:239-252 (1999). CPP was measured through a sidearm in the Langendorff system. Coronary vascular resistance (CVR) was defined as CPP divided by coronary flow rate. Hearts were perfused with Tat-conjugated δV1-1 [50 nmol/L; Tat-conjugated δV1-1 described in Chen, L. et al., Proc. Natl. Acad. Sci. U.S.A. 98:11114-11119 (2001)] or vehicle (control) during the first 20 minutes of reperfusion (n=5 for each group). Coronary perfusion effluent was collected to determine creatine phosphokinase (CPK) release.

### Immunohistochemistry and Histomorphometry

At the end of the reperfusion, 1 -mm-thick transverse sections of mouse hearts were incubated in triphenyltetrazolium chloride solution (TTC) (1% in phosphate buffer, pH 7.4) at 37° C. for 15 minutes as described in Inagaki, K. et al., Circulation 108:869-875 (2003) to determine the viable myocardium. One cm-thick transverse segments of the hearts were stained with TTC. Infarct size was expressed as a percentage of the total area at risk. Immunohistochemistry was performed on cardiac tissue two hours after reperfusion in murine hearts (n=5 for each group) as described in Vakeva, A. P., et al., Circulation 97:2259-2267 (1998). Hearts were immediately frozen in Optimal Cutting Temperature (OCT) Compound, and 5-μm-thick cryosections were obtained. Sections were fixed with 4% formaldehyde, blocked with 1 % normal donkey serum and incubated with mouse monoclonal anti-(x-actinin antibody (Sigma-Aldrich) or goat anti-PECAM-1 antibody (Santa Cruz Biochemicals) to distinguish between endothelial cells and myocytes. Secondary antibody treatments were carried out using goat anti-mouse IgG antibody conjugated with FITC or donkey anti-goat lgG antibody conjugated with FITC (Molecular Probe). Terminal deoxynucleotidyl transferase-mediated dUTP nick-end labeling (TUNEL) staining was performed for detection of apoptotic cells (Roche) and nuclei were counterstained with 4, 6-diamidino-2-phenylindole (DAPI; Sigma-Aldrich). Tissue samples from the non-ischemic area were used as negative control. TUNEL-positive nuclei were counted in a total of 1,500 myocytes and 500 of endothelial cells over several random fields.

### Statistical Analysis

Data are expressed as mean±SEM. Two-way ANOVA for repeated measures was used for time-course of cardiac function and vascular function, 1-way factorial ANOVA with Fisher's test for multiple comparisons, and unpaired or paired student's t-test for difference between 2 groups. P<0.05 was considered statistically significant.

### Results

Using transgenic mice expressing δPKC inhibitor only in their cardiomyocytes and wild type mice, ischemia/reperfusion damage was determined in vascular endothelial cells and cardiomyocytes with and without further exogenous δV1-1 infusion at reperfusion. In δV1-1-expressing hearts, infarct size and CPK release (FIGS. 1A-C) were decreased by 70% as compared to wild type mouse hearts. Delivery of δV1-1 through coronary arteries in wild type hearts also resulted in an about 70% decrease in infarct size and CPK release. Infarct size and CPK release were unaffected by further δV1-1 infusion to the transgenic hearts. However, although both transgenic and wild type mouse hearts had similar CVR at baseline, transgenic mouse hearts had a significantly lower CVR as compared to wild type mouse hearts during reperfusion and further treatment with δV1-1 significantly minimized the rise of CVR in transgenic mouse hearts (FIG. 1D).

To detebrmine whether exogenous δV1-1 treatment and/or selective expression of δV1-1 in myocytes prevents reperfusion-induced apoptosis of both endothelial cells and myocytes, TUNEL staining was performed on heart tissues after ischemia/reperfusion (FIG. 1 E, F). In wild type hearts, exogenous δV1-1 reduced the number of TUNEL positive endothelial cells and myocytes by 80%. In transgenic mouse hearts, expression of δV1-1 resulted in lower number of TUNEL-positive myocytes, but did not prevent apoptosis in endothelial cells; treatment with exogenous δV1-1 decreased TUNELpositive endothelial cells by 80% without any further effect on myocytes. Because the expression of δV1-1 in myocytes did not prevent apoptosis in endothelial cells, but decreased CVR during reperfusion, the decrease in CVR may occur via inhibition of myocytes swelling.

### EXAMPLE 2

The Effect of In vivo Treatment with 8PKC Inhibitor on Microvascular Function After Reperfusion Using a Porcine Model of Acute Myocardial Infarction (AMI)

The present example shows that in vivo treatment with δV1-1 preserves microvascular function and cardiac function after reperfusion in a porcine model of AMI.

### Methods

In vivo Porcine Model of Regional Ischemia and Local Peptide Delivery

Yorkshire swine (30-45kg) were maintained during the every procedure under anesthesia by inhaled isoflurane (1-2%). A bolus of 300 IU/kg heparin was administered intravenously through the sheath (6 French) placed in the carotid artery. A 10 mm, over-the-wire angioplasty balloon was placed in the left anterior descending artery (LAD) proximal to the first diagonal branch. The balloon was inflated to occlude the LAD for 30 minutes. At the last 1 minute of a 30-minute ischemia, Tat-conjugated δV1-1 (250 ng/kg) or saline was infused at 1 mL/minute for 1 minute through the guide-wire lumen of the balloon catheter (n=9 for each group). Left ventriculogram (LVG) was obtained (40° left anterior oblique projection, 30 frames/sec) to measure cardiac function at 5 time points; before ischemia (baseline), 30 minutes, 24 hours, 5 days, and 10 days after ischemia (n=9 for each group). Ejection fraction (EF) and hypokinetic area were calculated using the software (Plus Plus, Sanders Data System, Calif.) with elimination of frames after premature ventricular contraction beats. Blood pressure and heart rate were measured just before LVG measurements at each time point through the water-filled catheter (Table 1).

### Coronary Flow Reserve

Coronary flow was measured by a 0.014″ Doppler-tipped guide wire (Flowire, JOMED Inc.) in the LAD, and the unaffected, left circumflex artery (LCx). The wire tip was placed 2 cm distal from the balloon-occluded site in the LAD. After stable baseline flow velocity was recorded, adenosine [endothelium-independent vasodilator; 48 μg; Suryapranata, H., et al., Circulation 89:1109-1117 (1994)] was infused intracoronarily to induce hyperemia (a transient increase in coronary blood flow through microvascular vasodilation) at 5 time points: before ischemia (baseline), 30 minutes, 24 hours, 5 days, and 10 days after ischemia (n=9 for each group). Bradykinin [endothelium-dependent vaodilator, 0.2ml of a 3×10⁻⁶M in saline; Rodriguez-Sinovas, A., et al., J. Appl. Physiol. 95:81-88 (2003)] was also infused intracoronary before ischemia and 24 hours after reperfusion (n=6 for each group). Coronary flow reserve was calculated by dividing the average peak velocity (APV) at hyperemic phase by the baseline APV as described in Suryapranata, H., et al., Circulation 89:1109-1117 (1994).

### Immunohistochemistry and Histomorphometry

Immunohistochemistry was performed on cardiac tissue as described in Example 1 with the exception that immunohisotchemistry was performed 4 hours after reperfusion in porcine hearts (n=3 for each group).

After reperfusion, to determine the area at risk in porcine hearts, LAD ligation was performed at the balloon-occluded site and Evan's Blue (0.0025%) was perfused as previously described in Inagaki, K., et al., Circulation 2304-2307 (2003). All other methods were performed as described in Example 1.

### Results

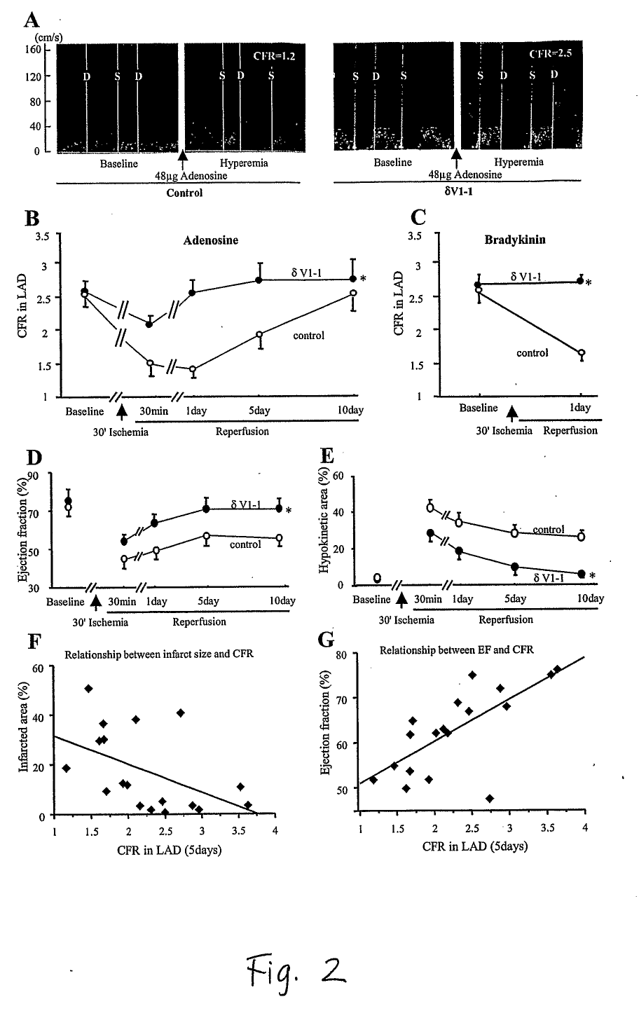

To further evaluate the effects of δV1-1 treatment in microvascular functions during the acute and recovery phases of reperfusion, the time-course of coronary flow reserve recovery in δV1-1-treated group was compared to that in control group using a porcine model of AMI. In control pigs, coronary flow reserve following adenosine infusion in LAD decreased significantly (2.5±0.2 to 1.5±0.1) 30 minutes after reperfusion, and did not fully recover to pre-ischemia level even 5 days after ischemia. In δV1-1-treated pigs, coronary flow reserve following adenosine infusion in LAD had a minor decrease and was normalized within 24 hours (Table 1, FIG. 2B).

TABLE 1
Hemodynamic data and CFR in the in vivo AMI porcine model.

| | Baseline | | 30 min R | | 24 hr R | |
|---|---|---|---|---|---|---|
| | δV1-1 | Control | δV1-1 | Control | δV1-1 | Control |
| HR, bpm | 98 ± 2 | 97 ± 5 | 90 ± 3 | 89 ± 7 | 103 ± 11 | 92 ± 6 |
| BP (Sys/Dia), mmHg | 92 ± 2/61 ± 4 | 95 ± 2/69 ± 3 | 87 ± 4/57 ± 3 | 89 ± 3/65 ± 3 | 101 ± 8/66 ± 13 | 96 ± 10/68 ± 13 |
| CFR(LAD) | 2.52 ± 0.16 | 2.46 ± 0.17 | 2.00 ± 0.20* | 1.51 ± 0.12† | 2.50 ± 0.24* | 1.44 ± 0.05† |
| b-APV, cm/s | 24 ± 3 | 26 ± 2 | 27 ± 4 | 28 ± 3 | 18 ± 1 | 18 ± 1 |
| h-APV, cm/s | 58 ± 7 | 63 ± 6 | 49 ± 4 | 39 ± 4† | 46 ± 6* | 27 ± 1† |
| CFR(Cx) | 2.44 ± 0.10 | 2.56 ± 0.16 | 1.73 ± 0.21 | 1.99 ± 0.18 | 1.96 ± 0.04 | 1.98 ± 0.15 |
| b-APV, cm/s | 24 ± 2 | 27 ± 2 | 20.3 ± 3 | 22 ± 2 | 15 ± 2 | 16 ± 2 |
| h-APV, cm/s | 61 ± 3 | 69 ± 5 | 37 ± 3 | 43 ± 4 | 30 ± 4 | 32 ± 3 |

| | |
|---|---|
| 5 days R | 10 days R |

| | δV1-1 | Control | δV1-1 | Control |
|---|---|---|---|---|
| HR, bpm | 98 ± 6 | 92 ± 4 | 90 ± 7 | 94 ± 4 |
| BP (Sys/Dia), mmHg | 97 ± 5/68 ± 3 | 99 ± 3/71 ± 3 | 94 ± 6/60 ± 3 | 90 ± 3/64 ± 4 |
| CFR(LAD) | 2.68 ± 0.21* | 1.81 ± 0.51† | 2.67 ± 0.27 | 2.24 ± 0.20 |
| b-APV, cm/s | 24 ± 3 | 27 ± 3 | 25 ± 4 | 29 ± 5 |
| h-APV, cm/s | 67 ± 9* | 47 ± 4† | 57 ± 6 | 62 ± 6 |
| CFR(Cx) | 2.01 ± 0.14 | 2.44 ± 0.17 | 2.46 ± 0.20 | 2.44 ± 0.21 |
| b-APV, cm/s | 25 ± 5 | 20 ± 3 | 25 ± 3 | 26 ± 4 |
| h-APV, cm/s | 55 ± 10 | 55 ± 10 | 55 ± 5 | 62 ± 9 |

R, reperfusion; b, baseline; h, hyperemia; APV, average peak flow velocity.
*p < 0.05 vs. control.
†p < 0.05 vs. Baseline

Bradykinin was infused to determine the effect of an endothelium dependent vasodilator in this porcine model. In control animals, coronary flow reserve in LAD following bradykinin infusion decreased significantly (2.7±0.1 to 1.6±0.1) 24 hours after reperfusion. However, in the δV1-1-treated group, following bradykinin infusion, coronary flow reserve did not decrease 24 hours (FIG. 2C). In all of the experiments, the resting APV and coronary flow reserve in left circumflex artery (LCx, control artery) remained normal at all time points in both groups. Similarly, there were no significant differences between the two groups in blood pressure, heart rate or vessel diameter (measured by intravascular ultrasound; data not shown), before and after intracoronary adenosine or bradykinin infusion.

In studies performed herein, intracoronarily treatment with δV1-1 at the time of reperfusion significantly lowered infarct size (30.2±4.5 vs. 4.4±1.1 %, P<0.001; control vs. δV1-1, n=9 for each group), improved ejection fraction (55.3±1.9 vs. 69.9±1.6%, P<0.05), and decreased hypokinetic area (24.9±4.0 vs. 4.7±2.0%, P<0.05) 10 days after ischemia (FIGS. 2D, E). A correlation was found between coronary flow reserve 5 days after reperfusion and infarct size (r=−0.49 P<0.05, n=18), and EF (r=0.7, P<0.05, n=18) 10 days after reperfusion (FIGS. 2F, G).

### EXAMPLE 3

Pathological Evidence for Protection from Reperfusion Injury by δV1-1 in the Porcine Model

This example shows that δV1-1 protects pigs from reperfusion injury. It specifically shows that, in a porcine model of AMI, δV1-1 treatment resulted in decreased apoptosis in endothelial cells and myocytes, decreased endothelial cell swelling, decreased myocyte damage and decreased red and white blood cell plugging of the capillary lumen.

### Methods

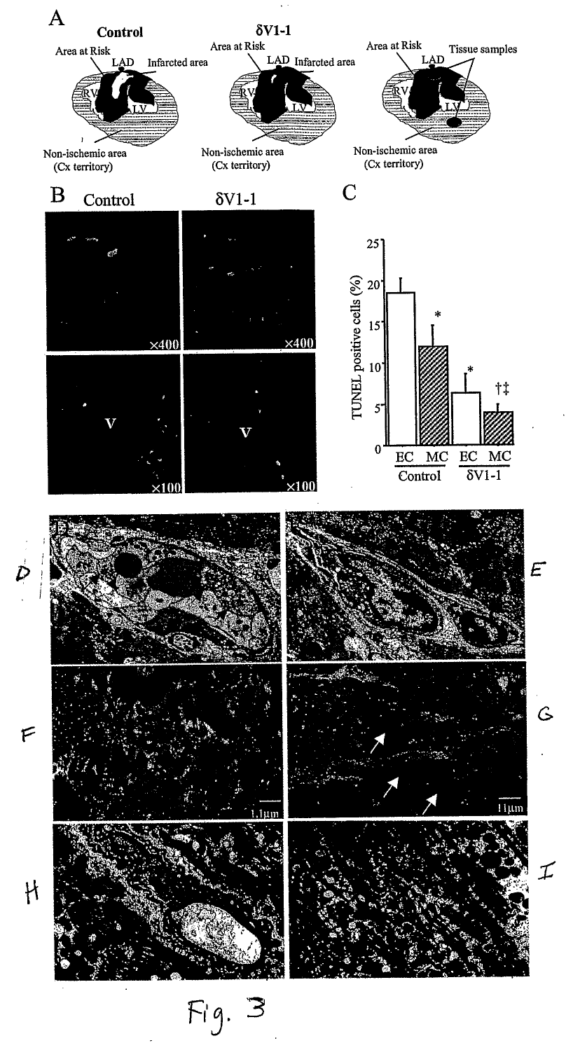

Electron microscopy study was performed on porcine cardiac tissue 4 hours after reperfusion (n=3 for each group). Samples taken from the mid-myocardium were fixed (FIG. 3A) and prepared as previously reported in Gottlieb, R. A., et al., J. Clin Invest. 94:1621-1628 (1994). Ultra-thin sections were stained with uranyl acetate lead citrate and examined with the H300 (Hitachi) electron microscopy. All other methods were as previously described in Examples 1 and 2.

### Results

Four hours after reperfusion, there was higher percentage of apoptosis in endothelial cells than in myocytes in the infarct territory of control hearts (18±2 vs.12±3%) (FIG. 3B, C). δV1-1 treatment decreased apoptosis in both endothelial cells and myocytes by about 70%. In hearts of control animals, there were red and white blood cells plugging the capillary lumen (FIG. 3D) and evidence of endothelial cells swelling and morphological hallmarks of apoptotic cell death, such as chromatin condensation and margination, were also observed in control hearts (FIG. 3E).

In contrast, in δV1-1-treated pigs, the endothelial cells exhibited minimal swelling and occasional endothelial folds. However, obstruction of capillaries by red and white blood cells invariably present in control hearts, was rarely seen in δV1-1-treated hearts (FIG. 3H). Further, in control pigs, myocyte damage was demonstrated by the appearance of contraction bands and swollen mitochondria with disrupted cristae and amorphous matrix densities in the ischemic zone (FIGS. 3F, G). In contrast, no such pathological changes were observed in δV1-1-treated hearts (FIGS. 3H, I).

### Discussion

To attain the full benefit from early restoration of blood flow, ischemic myocardium has to be protected against reperfusion injury that may be induced after reestablishment of flow. Braunwald, E. and Klaoner, R. A., J. Clin Invest. 76:1713-1719 (1985). Reperfusion itself exacerbates microvascular dysfunction when blood flow was restored to the infarct region. Braunwald, E. and Klaoner, R. A., J. Clin Invest. 76:1713-1719 (1985). The structural damage of the microvasculature prevents restoration of normal blood flow to the cardiac myocytes, which leads to inadequate healing of the cardiac scar and may prevent the development of future collateral flow. Several pharmacological approaches have been investigated, although none has demonstrated any significant clinical cardioprotective effects as discussed in Yellon, D. M., and Baxter, G. F., et al., Heart 83:381-387 (2000). Importantly, the current study indicates that reperfusion injury causes microvasculature damage that was prevented when δV1-1 was injected at reperfusion. The results presented here support the contribution of the microvascular damage/dysfunction to the outcome following an ischemic event.

The no-reflow phenomenon, a manifestation of microvascular damage, impedes normal blood flow to a vulnerable area after the main occlusion in the coronary arteries has been removed. No-reflow is observed in about 30% of patients with a reperfused anterior wall acute myocardial infarction (AMI) [Ito, H., et al., Circulation 93:223-228 (1996)] and is Associated with malignant arrhythmias, lower ejection fraction, or more cardiac death as discussed in Ito, H., et al., Circulation 93:223-228 (1996); Rezkalla, S. H. and Kloner, R. A. Circulation 105:656-662 (2002); and Morishima, I., et al., J. Am. Coll. Cardiol. 36:1202-1209 (2000). Potential mechanisms of no-reflow include endothelial swelling and protrusions, leukocyte plugging, microvascular dysfunction and mechanical compression of vasculature by myocardial swelling as discussed in Kloner, R. A. et al., J. Clin. Invest. 54:1496-1508 (1974) and Reffelmann, T. and Kloner, R. A. Heart 87:162-168 (2002). Preventing this damage enhances delivery of blood to the ischemic area, and thus reduces extension of infarct size as discussed in Rezkalla, S. H. and Kloner, R. A. Circulation 105:656-662 (2002). It is shown herein that δV1-1 improved coronary vascular function when administered at reperfusion by preserving the ultrastructure of both microvasculature and myocardium. Therefore, the δPKC inhibitor reduced infarct size and improved cardiac function, at least in part, by attenuating microvascular damage.

The occurrence of apoptosis in the myocardium following reperfusion has been already demonstrated in a number of species, including humans [Vakeva, A. P., et al., Circulation 97:2259-2267 (1998); and Gottlieb, R. A., et al. J. Clin. Invest. 94:1621-1628 (1994)]. δV1-1 inhibits hyperglycemia-induced apoptosis and free radical formation in adult rat cardiomyocytes [Shizukuda, Y. et al. Am. J. Physiol. Heart. Circ. Physiol. 282:H1625-1634 (2002)]. In another study, vascular cells from δPKC knockout mice have increased resistance to apoptosis due to reduction in free radical generation and mitochondrial dysfunction in response to stress stimuli [Leitges, M. et al., J. Clin. Invest. 108:1505-1512 (2001). Here, the application of δPKC inhibitor during reperfusion inhibited apoptosis in both endothelial cells and myocytes.

It is shown herein that in the hearts expressing δV1-1 in myocytes only, further treatment with δV1-1 delivered through the coronary arteries reduced apoptosis in endothelial cells and improved vascular function, but did not confer any additive protective effects in myocytes. These data suggest that δPKC is activated independently in endothelial cells and myocytes resulting in apoptosis. As shown in FIG. 1D, the expression of δV1-1 in cardiac myocytes reduced coronary vascular resistance (FIG. 1D), but did not prevent apoptosis of endothelial cells (FIG. 1E). Furthermore, if δV1-1 peptide was released from myocytes and then δV1-1 peptide protected endothelial cells, released δV1-1 should reduce apoptosis in endothelial cells. It is therefore suggest herein that the expression of δV1-1 in myocytes decreased coronary vascular resistance through inhibiting myocytes swelling, not by directly protecting endothelial cells.

Evidence from some studies support that inhibition of apoptosis reduces reperfusion injury. Recent studies demonstrate that the pan-caspase inhibitor (ZVADfmk) reduces reperfusion injury in in vivo rat myocardium [Yaoita, H. et al., Circulation 97:276-281 (1998)], and also reduces staurosporin-induced endothelial apoptosis, which followed by vessel thrombosis and endothelial denudation in in vivo rabbit femoral arteries [Durand, E. et al., Circulation 109:2503-2506 (2004)]. Furthermore, δPKC regulates caspase-3 activity [Kaul, s. et al., Eur. J. Neurosci. 18:1387-1401 (2003)]; caspase-3 activity is attenuated by δPKC inhibitor (rottlerin) and catalytically active recombinant δPKC increases caspase-3 activity [Kaul, s. et al., Eur. J. Neurosci. 18:1387-1401 (2003)]. Previous work herein also showed that δPKC inhibitor, δV1-1, reduced reperfusion-induced caspase-3 activity in myocardium [Inagaki, K. et al., Circulation 108:2304-2307 (2003)]. Thus, a δPKC inhibitor or a caspase inhibitor inhibits apoptosis in vascular endothelial cells and in cardiomyocytes following ischemia and these should be potent agents for inhibiting reperfusion injury.

The controversy as to the role of PKC isozymes in ischemia/reperfusion remains, at least in part, due to the use of isozymes-non-selective tools [Brooks, G., and Hearse, D. J., Circ. Res. 79:627-630 (1996). It was found herein that δPKC mediates reperfusion injury in this study. In contrast, some earlier studies suggested that δPKC plays a cardioprotective role in ischemic preconditioning [Kawamura, S. et al., Am. J. Physiol. 275:H2266-2271 (1998); Zhao, J. et al., J. Biol. Chem. 273:23072-23079 (1998)]. However, in those studies δPKC activation was induced before the ischemic event, not during reperfusion. In addition, εPKC was also activated, and may have contributed to the cardioprotection [Chen, L., et al., Proc. Natl. Acad. Sci. U.S.A. 98:11114-11119 (2001); Inagaki, K. et al., Circulation 108:869-875 (2003)]. Furthermore, it has also been shown that δPKC activation an hour prior to the ischemic event induced εPKC activation via adenosine A1 receptor (Inagaki et al. submitted). Therefore, the seemingly conflicting reports on the role of individual PKC isozymes in ischemia/reperfusion may reflect the non-specific pharmacological tools used as well as the timing of drug application to study ischemia/reperfusion.

In conclusion, administration of a δPKC-specific inhibitor for one minute at the onset of reperfusion improves microvascular function by reducing apoptotic cell death in vascular endothelial cells and occlusion of the microvasculature due to endothelial cell swelling and/or cellular plugging of the vessel. These data suggest that such a δPKC-specific inhibitor may be a potent therapeutic agent for reperfusion injury in patients with acute myocardial infarction.

The invention has been described above in detail, with specific reference to its preferred embodiments. It will be understood, however, that a variety of modifications and additions can be made to the invention disclosed without departing from the spirit and scope of the invention. Such modifications and additions are desired to be protected. In addition, all references cited herein are indicative of the level of skill in the art and are hereby incorporated by reference in their entirety.

What is claimed is:

1. A method of decreasing the extent of occlusion in the lumen of a mammalian blood vessel due to an ischemic event, comprising:
administering to a patient in need thereof a therapeutically effective amount of an inhibitor of δ protein kinase C, wherein said blood vessel is a blood vessel of the microvasculature.

2. The method of claim 1, wherein said inhibitor is a peptide.

3. The method of claim 2, wherein said peptide is δV1-1 having an amino acid sequence set forth in SEQ ID NO:1.

4. The method of claim 2, wherein said peptide is δV1-1 having an amino acid sequence set forth in SEQ ID NO:1, δV1-2 having an amino acid sequence set forth in SEQ ID NO:2, δV1-5 having an amino acid sequence set forth in SEQ ID NO:3, δV5 having an amino acid sequence set forth in SEQ ID NO:4, or a combination thereof.

5. The method of claim 2, wherein said peptide is δV1-1 having an amino acid sequence set forth in SEQ ID NO:1, δV1-2 having an amino acid sequence set forth in SEQ ID NO:2, δV1-5 having an amino acid sequence set forth in SEQ ID NO:3, δV5 having an amino acid sequence set forth in SEQ ID NO:4, a fragment of δV1-1, a fragment of δV1-2, a fragment of δV1-5, a fragment of δV5, a derivative of δV1-1, a derivative of δV1-2, a derivative of δV1-5, a derivative of δV5, or a combination thereof.

6. The method of claim 2, wherein said peptide has an amino acid sequence having at least about 50% identity to the amino acid sequence of δV1-1 set forth in SEQ ID NO:1, at least about 50% identity to the amino acid sequence of δV1-2 set forth in SEQ ID NO:2, at least about 50% identity to the amino acid sequence of δV1-5 set forth in SEQ ID NO:3, or at least about 50% identity to the amino acid sequence of δV5 set forth in SEQ ID NO:4.

7. The method of claim 1, wherein said blood vessel is a capillary, arteriole or venule.

8. The method of claim 7, wherein said capillary has an inner diameter of about 5 μm to about 10 μm.

9. The method of claim 1, wherein said occlusion is further caused by reperfusion-induced injury to said blood vessel.

10 The method of claim 1, wherein endothelial swelling contributes to said occlusion.

11. The method of claim 1, wherein said occlusion is caused by blood cells in said blood vessel.

12. The method of claim 11, wherein said blood cells are leukocytes, erythrocytes, or a combination thereof.

13. The method of claim 1, further comprising administering a second therapeutic agent

14. The method of claim 13, wherein said second therapeutic agent is a vasodilator.

15. The method of claim 14, wherein said vasodilator is bradykinin, adenosine, prostacyclin, iloprost, cisaprost; nicotinic acid, niacin, a beta adrenergic blocking drug, or a combination thereof.

16. A method of decreasing endothelial cell swelling in a mammalian blood vessel due to an ischemic event, comprising:
administering to a patient in need thereof a therapeutically effective amount of an inhibitor of δ protein kinase C.

17. The method of claim 16, wherein said inhibitor is a peptide.

18. The method of claim 16, wherein said peptide is δV1-1 having an amino acid sequence set forth in SEQ ID NO:1.

19. The method of claim 16, wherein said peptide is δV1-1 having an amino acid sequence set forth in SEQ ID NO:1, δ1-2 having an amino acid sequence set forth in SEQ ID NO:2, δV1-5 having an amino acid sequence set forth in SEQ ID NO:3, δV5 having an amino acid sequence set forth in SEQ ID NO:4, or a combination thereof.

20. The method of claim 16, wherein said peptide is δV1-1 having an amino acid sequence set forth in SEQ ID NO:1, δV1-2 having an amino acid sequence set forth in SEQ ID NO:2, δV1-5 having an amino acid sequence set forth in SEQ ID NO:3, δV5 having an amino acid sequence set forth in SEQ ID NO:4, a fragment of δV1-1, a fragment of δV1-2, a fragment of δV1-5, a fragment of δV5, a derivative of δV1-1, a derivative of δV1-2, a derivative of δV1-5, a derivative of δV5, or a combination thereof.

21. The method of claim 16, wherein said peptide has an amino acid sequence having at least about 50% identity to the amino acid sequence of δV1-1 set forth in SEQ ID NO:1, at least about 50% identity to the amino acid sequence of δV1-2 setforth in SEQ ID NO:2, at least about 50% identity to the amino acid sequence of δV1-5 set forth in SEQ ID NO:3, or at least about 50% identity to the amino acid sequence of δV5 set forth in SEQ ID NO:4.

22. The method of claim 16, wherein said blood vessel is a capillary.

23. The method of claim 22, wherein said capillary has an inner diameter of about 5 μm to about 10 μm.

24. The method of claim 14, further comprising administering a second therapeutic agent.

25. The method of claim 24, wherein said second therapeutic agent is a vasodilator.

26. The method of claim 25, wherein said vasodilator is bradykinin, adenosine, prostacyclin, iloprost, cisaprost; nicotinic acid, niacin, a beta adrenergic blocking drug, or a combination thereof.


SEQUENCE LISTING (85 sequences)

SEQ ID NO: 1 - protein, 10 aa, Rattus norvegicus
SFNSYELGSL

SEQ ID NO: 2 - protein, 11 aa, Rattus norvegicus
ALTTDRGKTLV

SEQ ID NO: 3 - protein, 14 aa, Rattus norvegicus
KAEFWLDLQPQAKV

SEQ ID NO: 4 - protein, 58 aa, Rattus norvegicus
PFRPKVKSPRPYSNFDQEFLNEKARLSYSDKNLIDSMDQSAFAGFSFVNPKFEHLLED

SEQ ID NO: 5 - protein, 10 aa, Artificial Sequence
  Synthetic peptide
TFNSYELGSL

SEQ ID NO: 6 - protein, 10 aa, Artificial Sequence
  Synthetic peptide
AFNSYELGSL

SEQ ID NO: 7 - protein, 10 aa, Artificial Sequence
  Synthetic peptide
SFNSYELGTL

SEQ ID NO: 8 - protein, 10 aa, Artificial Sequence
  Synthetic peptide
TFNSYELGTL

SEQ ID NO: 9 - protein, 10 aa, Artificial Sequence
  Synthetic peptide
SYNSYELGSL

SEQ ID NO: 10 - protein, 10 aa, Artificial Sequence
  Synthetic peptide
SFNSFELGSL

SEQ ID NO: 11 - protein, 9 aa, Artificial Sequence
  Synthetic peptide
SNSYDLGSL

SEQ ID NO: 12 - protein, 10 aa, Artificial Sequence
  Synthetic peptide
SFNSYELPSL

SEQ ID NO: 13 - protein, 10 aa, Artificial Sequence
  Synthetic peptide
SFNSYEIGSV

SEQ ID NO: 14 - protein, 10 aa, Artificial Sequence
  Synthetic peptide
SFNSYEVGSI

SEQ ID NO: 15 - protein, 10 aa, Artificial Sequence
  Synthetic peptide
SFNSYELGSV

SEQ ID NO: 16 - protein, 10 aa, Artificial Sequence
  Synthetic peptide
SFNSYELGSI

SEQ ID NO: 17 - protein, 10 aa, Artificial Sequence
  Synthetic peptide
SFNSYEIGSL

SEQ ID NO: 18 - protein, 10 aa, Artificial Sequence
  Synthetic peptide
SFNSYEVGSL

SEQ ID NO: 19 - protein, 10 aa, Artificial Sequence
  Synthetic peptide
AFNSYELGSL

SEQ ID NO: 20 - protein, 6 aa, Rattus norvegicus
YELGSL

SEQ ID NO: 21 - protein, 6 aa, Artificial Sequence
  Synthetic peptide
YDLGSL

SEQ ID NO: 22 - protein, 6 aa, Artificial Sequence
  Synthetic peptide
FDLGSL

SEQ ID NO: 23 - protein, 6 aa, Artificial Sequence
  Synthetic peptide
YDIGSL

SEQ ID NO: 24 - protein, 4 aa, Artificial Sequence
  Synthetic peptide
IGSL

SEQ ID NO: 25 - protein, 6 aa, Artificial Sequence
  Synthetic peptide
YDVGSL

SEQ ID NO: 26 - protein, 6 aa, Artificial Sequence
  Synthetic peptide
YDLPSL

SEQ ID NO: 27 - protein, 6 aa, Artificial Sequence
  Synthetic peptide
YDLGIL

SEQ ID NO: 28 - protein, 6 aa, Artificial Sequence
  Synthetic peptide
YDLGSI

SEQ ID NO: 29 - protein, 6 aa, Artificial Sequence
  Synthetic peptide
YDLGSV

SEQ ID NO: 30 - protein, 4 aa, Artificial Sequence
  Synthetic peptide
LGSL

SEQ ID NO: 31 - protein, 4 aa, Artificial Sequence
  Synthetic peptide
IGSL

SEQ ID NO: 32 - protein, 4 aa, Artificial Sequence
  Synthetic peptide
VGSL

SEQ ID NO: 33 - protein, 4 aa, Artificial Sequence
  Synthetic peptide
LPSL

SEQ ID NO: 34 - protein, 4 aa, Artificial Sequence
  Synthetic peptide
LGIL

SEQ ID NO: 35 - protein, 4 aa, Artificial Sequence
  Synthetic peptide
LGSI

SEQ ID NO: 36 - protein, 4 aa, Artificial Sequence
  Synthetic peptide
LGSV

SEQ ID NO: 37 - protein, 11 aa, Artificial Sequence
  Synthetic peptide
ALSTDRGKTLV

SEQ ID NO: 38 - protein, 11 aa, Artificial Sequence
  Synthetic peptide
ALTSDRGKTLV

SEQ ID NO: 39 - protein, 11 aa, Artificial Sequence
  Synthetic peptide
ALTTDRGKSLV

SEQ ID NO: 40 - protein, 11 aa, Artificial Sequence
  Synthetic peptide
ALTTDRPKTLV

SEQ ID NO: 41 - protein, 11 aa, Artificial Sequence
  Synthetic peptide
ALTTDRGRTLV

SEQ ID NO: 42 - protein, 11 aa, Artificial Sequence
  Synthetic peptide
ALTTDKGKTLV

SEQ ID NO: 43 - protein, 11 aa, Artificial Sequence
  Synthetic peptide
ALTTDKGKTLV

SEQ ID NO: 44 - protein, 14 aa, Artificial Sequence
  Synthetic peptide
RAEFWLDLQPQAKV

SEQ ID NO: 45 - protein, 14 aa, Artificial Sequence
  Synthetic peptide
KADFWLDLQPQAKV

SEQ ID NO: 46 - protein, 14 aa, Artificial Sequence
  Synthetic peptide
KAEFWLELQPQAKV

SEQ ID NO: 47 - protein, 14 aa, Artificial Sequence
  Synthetic peptide
KAEFWLDLQPQARV

SEQ ID NO: 48 - protein, 14 aa, Artificial Sequence
  Synthetic peptide
KAEYWLDLQPQAKV

SEQ ID NO: 49 - protein, 14 aa, Artificial Sequence
  Synthetic peptide
KAEFWIDLQPQAKV

SEQ ID NO: 50 - protein, 14 aa, Artificial Sequence
  Synthetic peptide
KAEFWVDLQPQAKV

SEQ ID NO: 51 - protein, 14 aa, Artificial Sequence
  Synthetic peptide
KAEFWLDIQPQAKV

SEQ ID NO: 52 - protein, 14 aa, Artificial Sequence
  Synthetic peptide
KAEFWLDVQPQAKV

SEQ ID NO: 53 - protein, 14 aa, Artificial Sequence
  Synthetic peptide
KAEFWLDLNPQAKV

SEQ ID NO: 54 - protein, 14 aa, Artificial Sequence
  Synthetic peptide
KAEFWLDLQPNAKV

SEQ ID NO: 55 - protein, 14 aa, Artificial Sequence
  Synthetic peptide
KAEFWLDLQPQAKI

SEQ ID NO: 56 - protein, 14 aa, Artificial Sequence
  Synthetic peptide
KAEFWLDLQPQAKI

SEQ ID NO: 57 - protein, 14 aa, Artificial Sequence
  Synthetic peptide
KAEFWADLQPQAKV

SEQ ID NO: 58 - protein, 14 aa, Artificial Sequence
  Synthetic peptide
KAEFWLDAQPQAKV

SEQ ID NO: 59 - protein, 14 aa, Artificial Sequence
  Synthetic peptide
KAEFWLDLQPQAKA

SEQ ID NO: 60 - protein, 7 aa, Rattus norvegicus
KAEFWLD

SEQ ID NO: 61 - protein, 8 aa, Rattus norvegicus
DLQPQAKV

SEQ ID NO: 62 - protein, 8 aa, Rattus norvegicus
EFWLDLQP

SEQ ID NO: 63 - protein, 7 aa, Rattus norvegicus
LDLQPQA

SEQ ID NO: 64 - protein, 7 aa, Rattus norvegicus
LQPQAKV

SEQ ID NO: 65 - protein, 7 aa, Rattus norvegicus
AEFWLDL

SEQ ID NO: 66 - protein, 7 aa, Rattus norvegicus
WLDLQPQ

SEQ ID NO: 67 - protein, 8 aa, Rattus norvegicus
SPRPYSNF

SEQ ID NO: 68 - protein, 8 aa, Rattus norvegicus
RPYSNFDQ

SEQ ID NO: 69 - protein, 8 aa, Rattus norvegicus
SNFDQEFL

SEQ ID NO: 70 - protein, 8 aa, Rattus norvegicus
DQEFLNEK

SEQ ID NO: 71 - protein, 8 aa, Rattus norvegicus
FLNEKARL

SEQ ID NO: 72 - protein, 8 aa, Rattus norvegicus
LIDSMDQS

SEQ ID NO: 73 - protein, 8 aa, Rattus norvegicus
SMDQSAFA

SEQ ID NO: 74 - protein, 8 aa, Rattus norvegicus
DQSAFAGF

SEQ ID NO: 75 - protein, 8 aa, Rattus norvegicus
FVNPKFEH

SEQ ID NO: 76 - protein, 8 aa, Rattus norvegicus
KFEHLLED

SEQ ID NO: 77 - protein, 8 aa, Rattus norvegicus
NEKARLSY

SEQ ID NO: 78 - protein, 8 aa, Rattus norvegicus
RLSYSDKN

SEQ ID NO: 79 - protein, 8 aa, Rattus norvegicus
SYSDKNLI

SEQ ID NO: 80 - protein, 8 aa, Rattus norvegicus
DKNLIDSM

SEQ ID NO: 81 - protein, 8 aa, Rattus norvegicus
PFRPKVKS

SEQ ID NO: 82 - protein, 8 aa, Rattus norvegicus
RPKVKSPR

SEQ ID NO: 83 - protein, 8 aa, Rattus norvegicus
VKSPRPYS

SEQ ID NO: 84 - protein, 17 aa, Artificial Sequence
  Drosophila antennapedia homeodomain-derived sequence
CRQIKIWFQNRRMKWKK

SEQ ID NO: 85 - protein, 11 aa, Human Immunodeficiency Virus, Type 1
YGRKKRRQRRR